(19) United States
(12) Patent Application Publication
(10) Pub. No.: US 2011/0003892 A1
(43) Pub. Date: Jan. 6, 2011

(54) ALL-TRANS RETINOID ESTERS AS ACTIVE PHARMACEUTICAL INGREDIENTS, ORAL AND TOPICAL DOSAGE FORM COMPOSITIONS THEREOF, AND METHODS OF TREATING SKIN CONDITIONS THEREOF

(76) Inventors: Margaret Clagett-Dame, Deerfield, WI (US); Hector F. Deluca, Deerfield, WI (US); Nirca J. Nieves, Madison, WI (US); Katarzyna Barycka, Madison, WI (US)

Correspondence Address: Intellectual Property Dept./Dewitt Ross & Stevens;Wisconsin Alumni Research Foundation, 2 East Mifflin Street,, Suite #600, Madison, WI, 53703-2865, US

(21) Appl. No.: 12/920,540

(22) PCT Filed: Mar. 6, 2009
(86) PCT No.: PCT/US09/36304

§ 371 Date: Sep. 1, 2010

Related U.S. Application Data
(60) Provisional application No. 61/034,391, filed on Mar. 6, 2008.

(51) Int. Cl. A61K 31/215; C07C 69/608; A61K 8/37; A61P 17/10; A61P 17/06; A61P 17/00; A61Q 19/08; A61Q 19/00

(52) U.S. Cl. 514/529; 560/128

(57) ABSTRACT

Active pharmaceutical ingredients being retinoic acid ester compounds including all- trans-retinoic acid tent-butyl ester, all-trans-retinoic acid iso-butyl ester, all-trans-retinoic acid iso-propyl ester, all-trans-retinoic acid sec-butyl ester, and, all-trans-retinoic acid 1-adamantyl ester, oral and topical dosage form compositions thereof, and methods of treating various skin conditions thereof.

### CROSS REFERENCE TO RELATED APPLICATIONS

This application claims priority to and benefit of U.S. Patent Application Ser. No. 61/034,391 filed on Mar. 6, 2008, which is incorporated herein by reference in its entirety.

U.S. Patent Application Publication No. US 2004/0167215 is commonly owned and discloses related subject matter.

U.S. Pat. No. 7,126,017 is commonly owned and discloses related subject matter.

### STATEMENT REGARDING GOVERNMENT INTEREST

Not applicable.

### BACKGROUND OF THE INVENTION

Retinoids are natural and synthetic compounds that are structurally related to vitamin A. All-trans retinol is the major circulating form of vitamin A. It is oxidized in the body to all-trans retinaldehyde, which can be further oxidized to all-trans retinoic acid (atRA). (Blomhoff et al., 1992, Annu. Rev. Nutr. 12:37-57; and, Moise et al., 2007, Biochemistry 46:4449-4458). atRA is the functional form of the vitamin that regulates growth, cellular differentiation, and embryonic development, whereas all-trans retinaldehyde functions in the visual cycle. (Clagett-Dame M et al., 2002, Annu. Rev. Nutr. 22:347-381).

Because atRA is such a potent regulatory molecule, it is formed in very limited amounts, and it is rapidly metabolized such that its half-life is relatively short. (Roberts et al., 1967, Biochem. J. 102:600-605). atRA is the endogenous ligand for the RAR family of receptors. The 13-cis retinoic acid isomer does not bind to the RARs. (Repa J J et al., 1993, Proc. Natl. Acad. Sci. USA 90:7293-7297).

atRA and other synthetic retinoids bind to and regulate the transcriptional activity of a family of nuclear proteins known as the retinoic acid receptors (“RARs”). atRA appears to act by binding to a series of RAR subtypes (α, β and γ), that also vary in sequence (isoforms) due to differential promoter usage and splicing. atRA and its analogs appear to bind to the nuclear RAR (α, β and γ) resulting in the regulation of target gene expression. (Chambon, 1996, FASEB J. 10:940-954; Clagett-Dame M et al., 1997, Crit. Rev. Euk. Gene Exp. 7:299-342; and, Mark et al., 2006, Annu. Rev. Pharmacol. Toxicol. 46:451-480). The 13-cis retinoic acid isomer must isomerize to atRA that is active in terms of receptor binding and activation.

Various forms of atRA and various synthetic retinoids have been used to treat a number of skin conditions, including acne, psoriasis, ichthyosis, photoaging, wrinkling, age spots and cancer, as well as to reduce skin atrophy caused by corticosteroid treatment for inflammatory diseases. (Fox L P et al., Goodman & Gilman's: The Pharmacological Basis of Therapeutics, Section XIII-Dermatology, 11th Ed.; Davidovici et al., 2007, Dermatol. Clin. 25:525-530). It has been estimated that 45 million people in the U.S. suffer from acne. Retinoids provide key therapeutic modalities in the treatment of acne. Topical and oral dosage forms of retinoids have been effective comedolytics. (Weiss et al., 2004, J. Drugs Dermatol. 3:146-154).

Acne is a condition of the pilosebaceous unit. Acne involves a spectrum of effects including non-inflammatory comedones, inflammatory papules, pustules and cysts. When administered topically or systemically, retinoids cause epidermal hyperproliferation leading to comedolysis and improvement of the disease. (Fisher G J et al., 1996, Molecular Mechanisms of Retinoid Actions in the Skin, FASEB. J. 10:1002:21013). Although very effective, retinoid therapy is substantially limited by the number and extent of side effects, which are particularly limiting when retinoids are administered orally.

Topical administration of retinoids has been limited largely due to side effects such as skin irritation (e.g., redness and burning), dryness and photosensitivity reactions. (Akhavan et al., 2003, Am. J. Clin. Dermatol. 4:473-492). However, topically administered retinoids have been a foundational treatment for many patients. (Zaenglein et al., Pediatrics 118:1188-1199, 2006).

The 13-cis form of atRA (i.e., isotretinoin, Accutane®) has been approved by the FDA for oral use to treat severe forms of acne. Accutane® has been approved for treating nodular acne by administering oral pharmacologic dosages of 0.5 to 2.0 mg/kg/day which inhibits sebaceous gland function and keratinization. Oral administration of 13-cis RA, however, induces an array of adverse side effects. Frank toxicity can lead to weight loss, bone loss, and liver toxicity; and with clinical use, perturbations in cholesterol, triglyceride and transaminase levels, and drying of mucosal membranes has been reported (Armstrong et al., 1994, The Retinoids, pp. 545-572; DiGiovanna, 2001, J. Am. Acad. Dermatol. 45:S176-S182). In addition, oral use of retinoid at clinically relevant concentrations can result in teratogenicity (Collins et al., 1999, Ann. Rev. Pharmacol. 39:399-430; Nau, 2001, J. Am. Acad. Dermatol. 45:S183-S187).

Systemic administration of retinoids has also been indicated for diseases such as psoriasis, pityriasis, rubra pilaris, condylomata accuminata, skin cancers, rosacea, hidradenitis, suppurativa, granuloma annular, lupus erythematosus and lichen planus. (Akyol M et al., 2006, Am. J. Clin. Derm. 6(3), 175-184).

### SUMMARY OF THE INVENTION

One aspect of the invention is an active pharmaceutical ingredient according to the structure

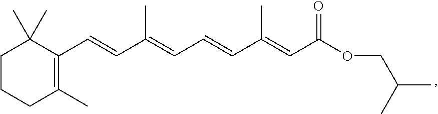

or, a solvate or hydrate thereof.

Another aspect of the invention is an active pharmaceutical ingredient according to the structure

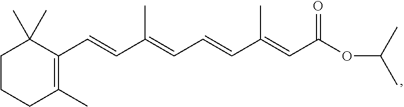

or, a solvate or hydrate thereof.

Another aspect of the invention is an active pharmaceutical ingredient according to the structure

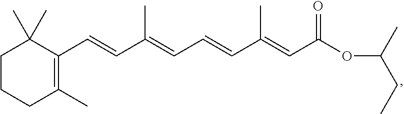

or, a solvate or hydrate thereof.

Another aspect of the invention is an active pharmaceutical ingredient according to the structure

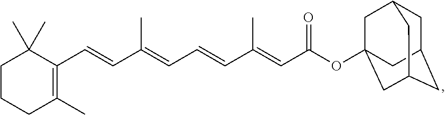

or, a solvate or hydrate thereof.

Another aspect of the invention is an active pharmaceutical ingredient according to the structure

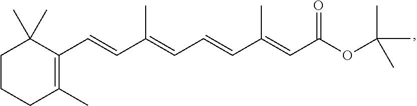

or, a solvate or hydrate thereof.

Another aspect of the invention is a method of treating acne comprising the acts or steps of orally administering a therapeutically effective dose of any one of the above active pharmaceutical ingredients to a human.

Another aspect of the invention is a method of treating acne comprising the acts or steps of topically administering a therapeutically effective dose of any one of the above active pharmaceutical ingredients to a human.

Another aspect of the invention is a method of reducing comedone size comprising the acts or steps of orally administering a therapeutically effective dose of any one of the above active pharmaceutical ingredients to a human.

Another aspect of the invention is a method of reducing comedone size comprising the acts or steps of topically administering a therapeutically effective dose of any one of the above active pharmaceutical ingredients to a human.

Another aspect of the invention is a method of treating psoriasis comprising the acts or steps of orally administering a therapeutically effective dose of any one of the above active pharmaceutical ingredients to a human.

Another aspect of the invention is a method of treating psoriasis comprising the acts or steps of topically administering a therapeutically effective dose of any one of the above active pharmaceutical ingredients to a human.

Another aspect of the invention is a method of treating ichthyosis comprising the acts or steps of orally administering a therapeutically effective dose of any one of the above active pharmaceutical ingredients to a human.

Another aspect of the invention is a method of treating ichthyosis comprising the acts or steps of topically administering a therapeutically effective dose of any one of the above active pharmaceutical ingredients to a human.

Another aspect of the invention is a method of treating photoaging comprising the acts or steps of orally administering a therapeutically effective dose of any one of the above active pharmaceutical ingredients to a human.

Another aspect of the invention is a method of treating photoaging comprising the acts or steps of topically administering a therapeutically effective dose of any one of the above active pharmaceutical ingredients to a human.

Another aspect of the invention is a method of treating skin atrophy caused by corticosteroid treatment comprising the acts or steps of orally administering a therapeutically effective dose of any one of the above active pharmaceutical ingredients to a human.

Another aspect of the invention is a method of treating skin atrophy caused by corticosteroid treatment comprising the acts or steps of topically administering a therapeutically effective dose of any one of the above active pharmaceutical ingredients to a human.

Another aspect of the invention is a method of treating photodamaged skin comprising the acts or steps of orally administering a therapeutically effective dose of any one of the above active pharmaceutical ingredients to a human.

Another aspect of the invention is a method of treating photodamaged skin comprising the acts or steps of topically administering a therapeutically effective dose of any one of the above active pharmaceutical ingredients to a human.

Another aspect of the invention is a method of increasing epidermal thickness comprising the acts or steps of orally administering a therapeutically effective dose of any one of the above active pharmaceutical ingredients to a human.

Another aspect of the invention is a method of increasing epidermal thickness comprising the acts or steps of topically administering a therapeutically effective dose of any one of the above active pharmaceutical ingredients to a human.

Another aspect of the invention is an oral dosage form composition comprising a therapeutically effective dose of an active pharmaceutical ingredient according to the structure

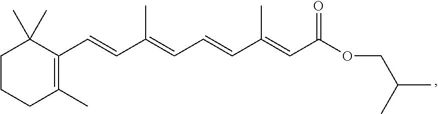

or, a solvate or hydrate thereof, and, a pharmaceutically suitable oral carrier system.

Another aspect of the invention is an oral dosage form composition comprising a therapeutically effective dose of an active pharmaceutical ingredient according to the structure

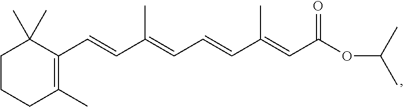

or, a solvate or hydrate thereof, and, a pharmaceutically suitable oral carrier system.

Another aspect of the invention is an oral dosage form composition comprising a therapeutically effective dose of an active pharmaceutical ingredient according to the structure

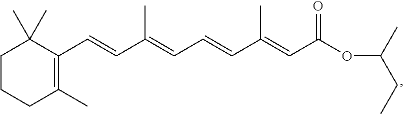

or, a solvate or hydrate thereof, and, a pharmaceutically suitable oral carrier system.

Another aspect of the invention is an oral dosage form composition comprising a therapeutically effective dose of an active pharmaceutical ingredient according to the structure

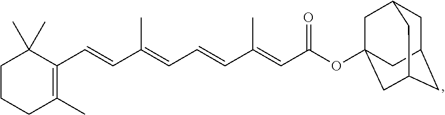

or, a solvate or hydrate thereof, and, a pharmaceutically suitable oral carrier system.

Another aspect of the invention is an oral dosage form composition comprising a therapeutically effective dose of an active pharmaceutical ingredient according to the structure

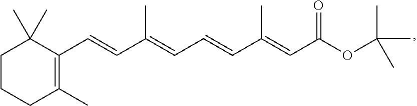

or, a solvate or hydrate thereof, and, a pharmaceutically suitable oral carrier system.

Another aspect of the invention is a topical dosage form composition comprising a therapeutically effective dose of an active pharmaceutical ingredient according to the structure

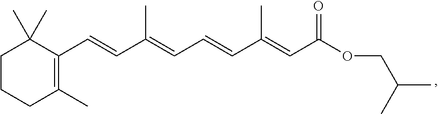

or, a solvate or hydrate thereof, and, a pharmaceutically suitable topical carrier system.

Another aspect of the invention is a topical dosage form composition comprising a therapeutically effective dose of an active pharmaceutical ingredient according to the structure

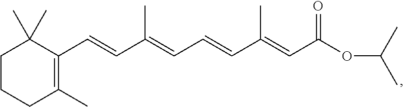

or, a solvate or hydrate thereof, and, a pharmaceutically suitable topical carrier system.

Another aspect of the invention is a topical dosage form composition comprising a therapeutically effective dose of an active pharmaceutical ingredient according to the structure

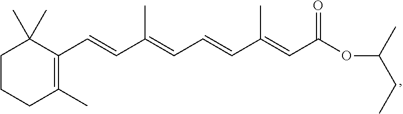

or, a solvate or hydrate thereof, and, a pharmaceutically suitable topical carrier system.

Another aspect of the invention is a topical dosage form composition comprising a therapeutically effective dose of an active pharmaceutical ingredient according to the structure

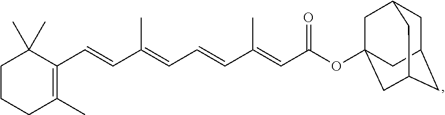

or, a solvate or hydrate thereof, and, a pharmaceutically suitable topical carrier system.

Another aspect of the invention is a topical dosage form composition comprising a therapeutically effective dose of an active pharmaceutical ingredient according to the structure

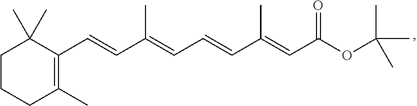

or, a solvate or hydrate thereof, and, a pharmaceutically suitable topical carrier system.

Another aspect of the invention is a method of treating acne comprising the acts or steps of orally administering any one of the above oral dosage form compositions to a human.

Another aspect of the invention is a method of treating acne comprising the acts or steps of topically administering any one of the above topical dosage form compositions to a human.

Another aspect of the invention is a method of reducing comedone size comprising the acts or steps of orally administering any one of the above oral dosage form compositions to a human.

Another aspect of the invention is a method of reducing comedone size comprising the acts or steps of topically administering any one of the above topical dosage form compositions to a human.

Another aspect of the invention is a method of treating psoriasis comprising the acts or steps of orally administering any one of the above oral dosage form compositions to a human.

Another aspect of the invention is a method of treating psoriasis comprising the acts or steps of topically administering any one of the above topical dosage form compositions to a human.

Another aspect of the invention is a method of treating ichthyosis comprising the acts or steps of orally administering any one of the above oral dosage form compositions to a human.

Another aspect of the invention is a method of treating ichthyosis comprising the acts or steps of topically administering any one of the above topical dosage form compositions to a human.

Another aspect of the invention is a method of treating photoaging comprising the acts or steps of orally administering any one of the above oral dosage form compositions to a human.

Another aspect of the invention is a method of treating photoaging comprising the acts or steps of topically administering any one of the above topical dosage form compositions to a human.

Another aspect of the invention is a method of treating skin atrophy caused by corticosteroid treatment comprising the acts or steps of orally administering any one of the above oral dosage form compositions to a human.

Another aspect of the invention is a method of treating skin atrophy caused by corticosteroid treatment comprising the acts or steps of topically administering any one of the above topical dosage form compositions to a human.

Another aspect of the invention is a method of treating photodamaged skin comprising the acts or steps of orally administering any one of the above oral dosage form compositions to a human.

Another aspect of the invention is a method of treating photodamaged skin comprising the acts or steps of topically administering any one of the above topical dosage form compositions to a human.

Another aspect of the invention is a method of increasing epidermal thickness comprising the acts or steps of orally administering any one of the oral dosage form compositions to a human.

Another aspect of the invention is a method of increasing epidermal thickness comprising the acts or steps of topically administering any one of the topical dosage form compositions to a human.

Exemplary embodiments of the invention are generally directed to active pharmaceutical ingredients being retinoic acid ester compounds including all-trans-retinoic acid tent-butyl ester, all-trans-retinoic acid iso-butyl ester, all-trans-retinoic acid iso-propyl ester, all-trans-retinoic acid sec-butyl ester, and, all-trans-retinoic acid 1-adamantyl ester, oral and topical dosage form compositions thereof, and methods of treating various skin conditions thereof.

All-trans-retinoic acid iso-butyl ester (referred to herein as IB-RA), all-trans-retinoic acid iso-propyl ester (referred to herein as IPE-RA), all-trans-retinoic acid sec-butyl ester (referred to herein as SB-RA), all-trans-retinoic acid tert-butyl ester (referred to herein as t-butyl-RA), and, all-trans-retinoic acid 1-adamantyl ester (referred to herein as 1A-RA) and methods of making each compound are disclosed herein and U.S. Pat. No. 7,126,017 and U.S. Patent Application Publication No. US 2004/0167215, which are both incorporated herein by reference. Solvates and hydrates can be made using conventional processes known in the art.

### BRIEF DESCRIPTION OF DRAWINGS OF EXEMPLARY EMBODIMENTS

FIG. 1 is a bar graph showing oral treatment of Rhino mice using SB-RA alone, IPE-RA alone, IB-RA alone, and t-butyl-RA alone producing a significant dose-dependent reduction in comedone area, whereby the comedone area was analyzed after 24 days of oral treatment with various doses of retinoid, whereby SB-RA, IPE-RA and IB-RA each produced a maximal reduction in comedone size as compared to the vehicle control at a dose measuring 26.2 μmole/kg_{BW}, and whereby the t-butyl-RA ester showed a maximal reduction in comedone size as compared to vehicle at a dose of 166 μmole/kg_{BW}.

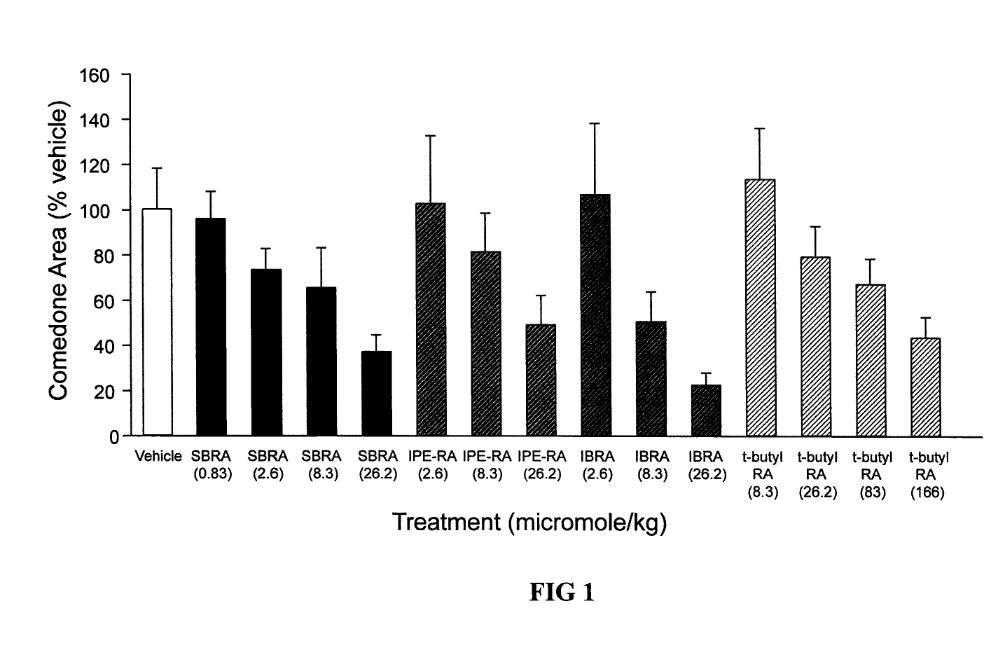

FIG. 2 is a bar graph showing oral treatment of Rhino mice using SB-RA alone, IPE-RA alone, IB-RA alone, and t-butyl-RA alone in terms of weight change from baseline, whereby the % change from the starting baseline weight was analyzed after 24 days of oral treatment with various doses of retinoid, and whereby there was no significant reduction in the overall group weight during the course of the study on any dose of retinoid studied.

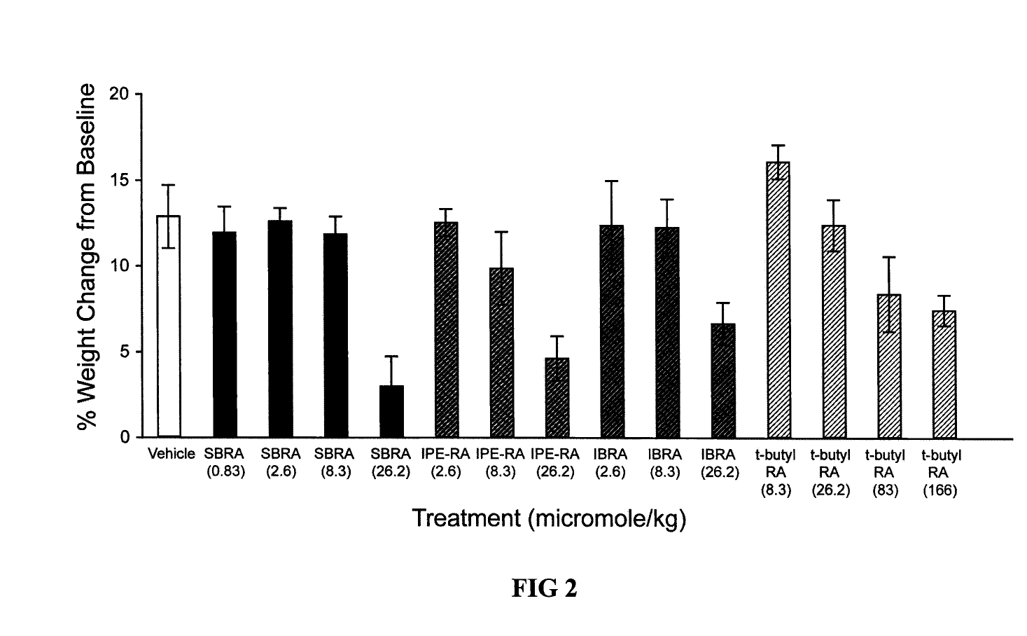

### DETAILED DESCRIPTION OF THE EXEMPLARY EMBODIMENTS

As used herein, “therapeutically effective dose” and “administering to a human a therapeutically effective dose” refers to an amount of one or more APIs sufficient to treat (e.g., prophylactic, treating the active condition or curing) one or more of acne vulgaris, psoriasis, ichthyosis, photoaging, photodamaged skin, skin cancer, and skin atrophy caused by corticosteroid treatment for inflammatory disease, and the like, as well as to reduce comedone size and increase epidermal skin thickness.

The pharmaceutically suitable topical and oral carrier systems (also referred to as drug delivery systems, which are modern technology, distributed with or as a part of a drug product that allows for the uniform release or targeting of drugs to the body) preferably include FDA-approved and/or USP-approved inactive ingredients. Under 21 CFR 210.3(b)(8), an inactive ingredient is any component of a drug product other than the active ingredient. According to 21 CFR 210.3(b)(7), an active ingredient is any component of a drug product intended to furnish pharmacological activity or other direct effect in the diagnosis, cure, mitigation, treatment, or prevention of disease, or to affect the structure or any function of the body of humans or other animals. Active ingredients include those components of the product that may undergo chemical change during the manufacture of the drug product and be present in the drug product in a modified form intended to furnish the specified activity or effect.

As used herein, the topical dosage form includes various dosage forms known in the art such as lotions (an emulsion, liquid dosage form, whereby this dosage form is generally for external application to the skin), lotion augmented (a lotion dosage form that enhances drug delivery, whereby augmentation does not refer to the strength of the drug in the dosage form), gels (a semisolid dosage form that contains a gelling agent to provide stiffness to a solution or a colloidal dispersion, whereby the gel may contain suspended particles), ointments (a semisolid dosage form, usually containing <20% water and volatiles 5 and >50% hydrocarbons, waxes, or polyols as the vehicle, whereby this dosage form is generally for external application to the skin or mucous membranes), ointment augmented (an ointment dosage form that enhances drug delivery, whereby augmentation does not refer to the strength of the drug in the dosage form), creams (an emulsion, semisolid dosage form, usually containing >20% water and volatiles 5 and/or <50% hydrocarbons, waxes, or polyols as the vehicle, whereby this dosage form is generally for external application to the skin or mucous membranes), cream augmented (a cream dosage form that enhances drug delivery, whereby augmentation does not refer to the strength of the drug in the dosage form), emulsion (a dosage form consisting of a two-phase system comprised of at least two immiscible liquids, one of which is dispersed as droplets, internal or dispersed phase, within the other liquid, external or continuous phase, generally stabilized with one or more emulsifying agents, whereby emulsion is used as a dosage form term unless a more specific term is applicable, e.g. cream, lotion, ointment), suspensions (a liquid dosage form that contains solid particles dispersed in a liquid vehicle), suspension extended release (a liquid preparation consisting of solid particles dispersed throughout a liquid phase in which the particles are not soluble; the suspension has been formulated in a manner to allow at least a reduction in dosing frequency as compared to that drug presented as a conventional dosage form, e.g., as a solution or a prompt drug-releasing, conventional solid dosage form), pastes (a semisolid dosage form, containing a large proportion, 20-50%, of solids finely dispersed in a fatty vehicle, whereby this dosage form is generally for external application to the skin or mucous membranes), solutions (a clear, homogeneous liquid 1 dosage form that contains one or more chemical substances dissolved in a solvent or mixture of mutually miscible solvents), powders, shampoos (a lotion dosage form which has a soap or detergent that is usually used to clean the hair and scalp; it is often used as a vehicle for dermatologic agents), shampoo suspensions (a liquid soap or detergent containing one or more solid, insoluble substances dispersed in a liquid vehicle that is used to clean the hair and scalp and is often used as a vehicle for dermatologic agents), aerosol foams (i.e., a dosage form containing one or more active ingredients, surfactants, aqueous or nonaqueous liquids, and the propellants; if the propellant is in the internal discontinuous phase, i.e., of the oil-in-water type, a stable foam is discharged, and if the propellant is in the external continuous phase, i.e., of the water-in-oil type, a spray or a quick-breaking foam is discharged), sprays (a liquid minutely divided as by a jet of air or steam), metered spray (a non-pressurized dosage form consisting of valves which allow the dispensing of a specified quantity of spray upon each activation), suspension spray (a liquid preparation containing solid particles dispersed in a liquid vehicle and in the form of coarse droplets or as finely divided solids to be applied locally, most usually to the nasal-pharyngeal tract, or topically to the skin), jellies (a class of gels, which are semisolid systems that consist of suspensions made up of either small inorganic particles or large organic molecules interpenetrated by a liquid—in which the structural coherent matrix contains a high portion of liquid, usually water), films (a thin layer or coating), film extended release (a drug delivery system in the form of a film that releases the drug over an extended period in such a way as to maintain constant drug levels in the blood or target tissue), film soluble (a thin layer or coating which is susceptible to being dissolved when in contact with a liquid), sponges (a porous, interlacing, absorbent material that contains a drug, whereby it is typically used for applying or introducing medication, or for cleansing, and whereby a sponge usually retains its shape), swabs (a small piece of relatively flat absorbent material that contains a drug, whereby a swab may also be attached to one end of a small stick, and whereby a swab is typically used for applying medication or for cleansing), patches (a drug delivery system that often contains an adhesive backing that is usually applied to an external site on the body, whereby its ingredients either passively diffuse from, or are actively transported from, some portion of the patch, whereby depending upon the patch, the ingredients are either delivered to the outer surface of the body or into the body, and whereby a patch is sometimes synonymous with the terms ‘extended release film’ and ‘system’), patch extended release (a drug delivery system in the form of a patch that releases the drug in such a manner that a reduction in dosing frequency compared to that drug presented as a conventional dosage form, e.g., a solution or a prompt drug-releasing, conventional solid dosage form), patch extended release electronically controlled (a drug delivery system in the form of a patch which is controlled by an electric current that releases the drug in such a manner that a reduction in dosing frequency compared to that drug presented as a conventional dosage form, e.g., a solution or a prompt drug-releasing, conventional solid dosage form), and the like. The various topical dosage forms may also be formulated as immediate release, controlled release, sustained release, or the like.

The topical dosage form composition contains an API and one or more inactive pharmaceutical ingredients such as excipients, colorants, pigments, additives, fillers, emollients, surfactants (e.g., anionic, cationic, amphoteric and nonionic), penetration enhancers (e.g., alcohols, fatty alcohols, fatty acids, fatty acid esters and polyols), and the like. Various FDA-approved topical inactive ingredients are found at the FDA's “The Inactive Ingredients Database” that contains inactive ingredients specifically intended as such by the manufacturer, whereby inactive ingredients can also be considered active ingredients under certain circumstances, according to the definition of an active ingredient given in 21 CFR 210.3(b)(7). Alcohol is a good example of an ingredient that may be considered either active or inactive depending on the product formulation.

As used herein, the oral dosage form includes capsules (a solid oral dosage form consisting of a shell and a filling, whereby the shell is composed of a single sealed enclosure, or two halves that fit together and which are sometimes sealed with a band, and whereby capsule shells may be made from gelatin, starch, or cellulose, or other suitable materials, may be soft or hard, and are filled with solid or liquid ingredients that can be poured or squeezed), capsule or coated pellets (solid dosage form in which the drug is enclosed within either a hard or soft soluble container or “shell” made from a suitable form of gelatin; the drug itself is in the form of granules to which varying amounts of coating have been applied), capsule coated extended release (a solid dosage form in which the drug is enclosed within either a hard or soft soluble container or “shell” made from a suitable form of gelatin; additionally, the capsule is covered in a designated coating, and which releases a drug or drugs in such a manner to allow at least a reduction in dosing frequency as compared to that drug or drugs presented as a conventional dosage form), capsule delayed release (a solid dosage form in which the drug is enclosed within either a hard or soft soluble container made from a suitable form of gelatin, and which releases a drug (or drugs) at a time other than promptly after administration, whereby enteric-coated articles are delayed release dosage forms), capsule delayed release pellets (solid dosage form in which the drug is enclosed within either a hard or soft soluble container or “shell” made from a suitable form of gelatin; the drug itself is in the form of granules to which enteric coating has been applied, thus delaying release of the drug until its passage into the intestines), capsule extended release (a solid dosage form in which the drug is enclosed within either a hard or soft soluble container made from a suitable form of gelatin, and which releases a drug or drugs in such a manner to allow a reduction in dosing frequency as compared to that drug or drugs presented as a conventional dosage form), capsule film-coated extended release (a solid dosage form in which the drug is enclosed within either a hard or soft soluble container or “shell” made from a suitable form of gelatin; additionally, the capsule is covered in a designated film coating, and which releases a drug or drugs in such a manner to allow at least a reduction in dosing frequency as compared to that drug or drugs presented as a conventional dosage form), capsule gelatin coated (a solid dosage form in which the drug is enclosed within either a hard or soft soluble container made from a suitable form of gelatin; through a banding process, the capsule is coated with additional layers of gelatin so as to form a complete seal), capsule liquid filled (a solid dosage form in which the drug is enclosed within a soluble, gelatin shell which is plasticized by the addition of a polyol, such as sorbitol or glycerin, and is therefore of a somewhat thicker consistency than that of a hard shell capsule; typically, the active ingredients are dissolved or suspended in a liquid vehicle), granule (a small particle or grain), pellet (a small sterile solid mass consisting of a highly purified drug, with or without excipients, made by the formation of granules, or by compression and molding), pellets coated extended release (a solid dosage form in which the drug itself is in the form of granules to which varying amounts of coating have been applied, and which releases a drug or drugs in such a manner to allow a reduction in dosing frequency as compared to that drug or drugs presented as a conventional dosage form), pill (a small, round solid dosage form containing a medicinal agent intended for oral administration), powder (an intimate mixture of dry, finely divided drugs and/or chemicals that may be intended for internal or external use), elixir (a clear, pleasantly flavored, sweetened hydroalcoholic liquid containing dissolved medicinal agents; it is intended for oral use), chewing gum (a sweetened and flavored insoluble plastic material of various shapes which when chewed, releases a drug substance into the oral cavity), syrup (an oral solution containing high concentrations of sucrose or other sugars; the term has also been used to include any other liquid dosage form prepared in a sweet and viscid vehicle, including oral suspensions), tablet (a solid dosage form containing medicinal substances with or without suitable diluents), tablet chewable (a solid dosage form containing medicinal substances with or without suitable diluents that is intended to be chewed, producing a pleasant tasting residue in the oral cavity that is easily swallowed and does not leave a bitter or unpleasant after-taste), tablet coated (a solid dosage form that contains medicinal substances with or without suitable diluents and is covered with a designated coating), tablet coated particles (a solid dosage form containing a conglomerate of medicinal particles that have each been covered with a coating), tablet delayed release (a solid dosage form which releases a drug or drugs at a time other than promptly after administration, whereby enteric-coated articles are delayed release dosage forms), tablet delayed release particles (a solid dosage form containing a conglomerate of medicinal particles that have been covered with a coating which releases a drug or drugs at a time other than promptly after administration, whereby enteric-coated articles are delayed release dosage forms), tablet dispersible (a tablet that, prior to administration, is intended to be placed in liquid, where its contents will be distributed evenly throughout that liquid, whereby term ‘tablet, dispersible’ is no longer used for approved drug products, and it has been replaced by the term ‘tablet, for suspension’), tablet effervescent (a solid dosage form containing mixtures of acids, e.g., citric acid, tartaric acid, and sodium bicarbonate, which release carbon dioxide when dissolved in water, whereby it is intended to be dissolved or dispersed in water before administration), tablet extended release (a solid dosage form containing a drug which allows at least a reduction in dosing frequency as compared to that drug presented in conventional dosage form), tablet film coated (a solid dosage form that contains medicinal substances with or without suitable diluents and is coated with a thin layer of a water-insoluble or water-soluble polymer), tablet film coated extended release (a solid dosage form that contains medicinal substances with or without suitable diluents and is coated with a thin layer of a water-insoluble or water-soluble polymer; the tablet is formulated in such manner as to make the contained medicament available over an extended period of time following ingestion), tablet for solution (a tablet that forms a solution when placed in a liquid), tablet for suspension (a tablet that forms a suspension when placed in a liquid, which is formerly referred to as a ‘dispersible tablet’), tablet multilayer (a solid dosage form containing medicinal substances that have been compressed to form a multiple-layered tablet or a tablet-within-a-tablet, the inner tablet being the core and the outer portion being the shell), tablet multilayer extended release (a solid dosage form containing medicinal substances that have been compressed to form a multiple-layered tablet or a tablet-within-a-tablet, the inner tablet being the core and the outer portion being the shell, which, additionally, is covered in a designated coating; the tablet is formulated in such manner as to allow at least a reduction in dosing frequency as compared to that drug presented as a conventional dosage form), tablet orally disintegrating (a solid dosage form containing medicinal substances which disintegrates rapidly, usually within a matter of seconds, when placed upon the tongue), tablet orally disintegrating delayed release (a solid dosage form containing medicinal substances which disintegrates rapidly, usually within a matter of seconds, when placed upon the tongue, but which releases a drug or drugs at a time other than promptly after administration), tablet soluble (a solid dosage form that contains medicinal substances with or without suitable diluents and possesses the ability to dissolve in fluids), tablet sugar coated (a solid dosage form that contains medicinal substances with or without suitable diluents and is coated with a colored or an uncolored water-soluble sugar), osmotic, and the like.

The oral dosage form composition contains an API and one or more inactive pharmaceutical ingredients such as diluents, solubilizers, alcohols, binders, controlled release polymers, enteric polymers, disintegrants, excipients, colorants, flavorants, sweeteners, antioxidants, preservatives, pigments, additives, fillers, suspension agents, surfactants (e.g., anionic, cationic, amphoteric and nonionic), and the like. Various FDA-approved topical inactive ingredients are found at the FDA's “The Inactive Ingredients Database” that contains inactive ingredients specifically intended as such by the manufacturer, whereby inactive ingredients can also be considered active ingredients under certain circumstances, according to the definition of an active ingredient given in 21 CFR 210.3(b)(7). Alcohol is a good example of an ingredient that may be considered either active or inactive depending on the product formulation.

As used herein, “hydrates” of the instant compound may be a pharmaceutically suitable (i.e., pharmaceutically acceptable) hydrate that is a compound formed by the addition of water or its elements to a host molecule (e.g., the free form version of the compound) including, but not limited to, monohydrates, dihydrates, etc.

As used herein, “solvates” of the instant compound may be a pharmaceutically suitable (i.e., pharmaceutically acceptable) solvate, whereby solvation is an interaction of a solute with the solvent which leads to stabilization of the solute species in the solution, and whereby the solvated state is an ion in a solution complexed by solvent molecules. Solvates and hydrates may also be referred to as “analogues.”

### Examples

Data for the API's being the all-trans-retinoic acid esters IB-RA, IPE-RA, SB-RA, and 1A-RA along with various synthesis data is set forth in Table 1 below.

TABLE 1

| Ester | ROH | Base | Yield |
|---|---|---|---|
| 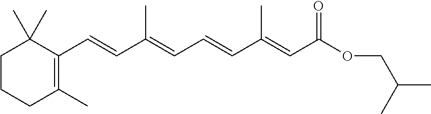 | iso-butanol 0.77 ml (0.61 g; 8.32 mmol) | pyridine 0.72 ml (0.695 g) (8.78 mmol) | 65% 0.771 g (2.16 mmol) |
| IB-RA | | | |
| 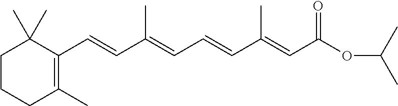 | iso-propanol 6.37 ml (5.00 g; 83.2 mmol) | Et₃N 1.22 ml (0.889 g) (8.78 mmol) | 75% 0.855 g (2.49 mmol) |
| IPE-RA | | | |
| 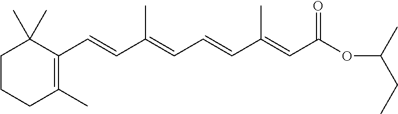 | sec-butanol 4.58 ml (3.70 g; 49.92 mmol) | Et₃N 1.22 ml (0.889 g) (8.78 mmol) | 83% 0.948 g (2.76 mmol) |
| SB-RA | | | |
| 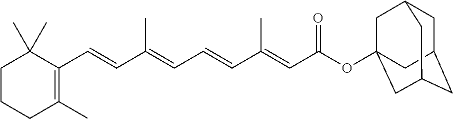 | 1-adamantanol 2.533 g (16.64 mmol) (in 15 ml of THF) | pyridine 1.28 ml (1.246 g) (15.96 mmol) | 32% 0.463 g (1.06 mmol) |
| 1A-RA | | | |

General procedure of the synthesis of all-trans-retinoic acid esters in accordance with Scheme 1.

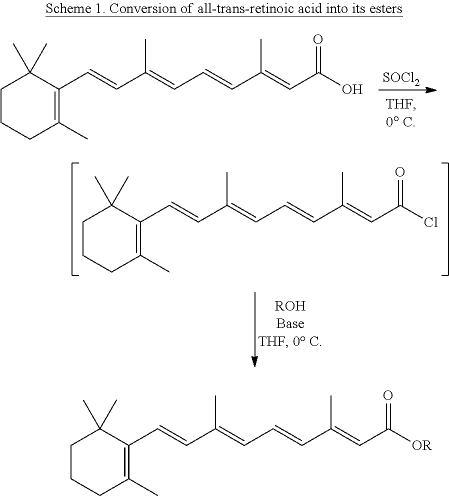

All-trans-retinoic acid (1.00 g; 3.33 mmol) was dissolved in anhydrous tetrahydrofuran (20 ml) at room temperature under argon in darkness. The solution was cooled to 0° C. and thionyl chloride (0.29 ml; 0.475 g; 3.99 mmol) was added dropwise to the vigorously stirred solution. After 1 hour, base and alcohol were added, and the reaction mixture turned to deep red suspension. When that reaction (compounds IB-RA, IPE-RA, SB-RA) was completed, diethyl ether (60 ml) was added, and the mixture was washed with water (3×15 ml) and brine (2×15 ml). Aqueous phase was washed with diethyl ether (2×10 ml), and the combined organic phases were dried over anhydrous MgSO₄. Drying agent was removed, and the solution was concentrated under reduced pressure. When reaction of compound 1A-RA was completed, solids were filtered off while washing with hexane (150 ml), and the filtrate was concentrated under reduced pressure. In all cases concerning compounds IB-RA, IPE-RA, SB-RA and 1A-RA, remainders were evaporated with hexane (3×10 ml), and the residues were purified by column chromatography (60:1 and 30:1 hexane/diethyl ether) to yield the respective end-products in the form of an oil.

All-trans-retinoic acid iso-butyl ester (IB-RA). ¹H NMR (400 MHz, CDCl₃): δ 0.94 (s, 3H), 0.96 (s, 3H), 2×1.03 (2×s, 2×3H), 1.47 and 1.61 (2×m, 2>2H), 1.71 (s, 3H), 1.98 (m, 2H), 1.99 (s, 3H), 2.35 (s, 3H), 3.90 (d, 2H, J=6.4 Hz), 5.79 (s, 1H), 6.13 (d, 1H, J=16.2 Hz), 6.14 (d, 1H, J=11.7 Hz), 6.27 (d, 1H, J=16.3 Hz), 6.28 (d, 1H, J=15.0 Hz), 6.99 (dd, 1H, J=11.5 Hz, J=15.0 Hz); ¹³C NMR (100 MHz, CDCl₃): δ 12.9, 13.9, 3×19.2, 21.7, 27.8, 2×28.9, 33.1, 34.3, 39.6, 69.9, 118.6, 128.6, 129.5, 129.9, 130.9, 135.2, 137.3, 137.7, 139.5, 152.6, 167.3; MS (ESI) exact mass calculated for C₂₄H₃₇O₂ ([M+H]⁺) 357.2793, found 357.2791.

All-trans-retinoic acid iso-propyl ester (IPE-RA). ¹H NMR (400 MHz, CDCl₃): δ 1.03 (2×s, 2×3H), 1.27 (d, 3H, J=6.3 Hz), 1.47 and 1.61 (2×m, 2×2H), 1.71 (s, 3H), 1.99 (s, 3H), 2.02 (m, 2H), 2.34 (s, 3H), 5.05 (sext, 1H, J=6.3 Hz, J=12.5 Hz), 5.74 (s, 1H), 6.13 (d, 1H, J=15.9 Hz), 6.14 (d, 1H, J=11.6 Hz), 6.27 (m, 2H), 6.98 (dd, 1H, J=11.4 Hz, J=15.0 Hz); ¹³C NMR (100 MHz, CDCl₃): δ 12.9, 13.8, 19.2, 21.7, 2×22.0, 2×28.9, 33.1, 34.3, 39.6, 66.8, 119.2, 128.5, 129.5, 129.9, 130.7, 135.3, 137.3, 137.7, 139.4, 152.3, 166.7; MS (ESI) exact mass calculated for C₂₃H₃₅O₂ ([M+H]⁺) 343.2637, found 343.2643.

All-trans-retinoic acid sec-butyl ester (SB-RA). ¹H NMR (500 MHz, CDCl₃): δ 0.92 (t, 3H, J=7.4 Hz), 2×1.03 (2×s, 2×3H), 1.23 (d, 3H, J=6.3 Hz), 1.47 (m, 2H), 1.60 (m, 3H), 1.71 (s, 3H), 2.00 (s, 3H), 2.20 (m, 2H), 2.35 (s, 3H), 4.89 (sext, 1H, J=6.3 Hz, J=12.5 Hz), 5.76 (s, 1H), 6.13 (d, 1H, J=16.0 Hz), 6.14 (d, 1H, J=11.5 Hz), 6.27 (d, 1H, J=16.1 Hz), 6.28 (d, 1H, J=15.1 Hz), 6.98 (dd, 1H, J=11.4 Hz, J=15.1 Hz); ¹³C NMR (125 MHz, CDCl₃): δ 9.76, 12.9, 13.8, 19.2, 19.6, 21.7, 3×28.9, 33.1, 34.2, 39.6, 71.3, 119.1, 128.5, 129.5, 129.9, 130.7, 135.3, 137.3, 137.7, 139.4, 152.3, 166.9; MS (ESI) exact mass calculated for C₂₄H₃₇O₂ ([M+H]⁺) 357.2793, found 357.2802.

All-trans-retinoic acid 1-adamantyl ester (1A-RA). ¹H NMR (400 MHz, CDCl₃): δ 1.02 (2×s, 2×3H), 1.47 and 1.60 (2×m, 2×2H), 1.68 (br s, 6H), 1.71 (s, 3H), 1.99 (s, 3H), 2.02 (m, 2H), 2.16 (s, 9H), 2.31 (s, 3H), 5.70 (s, 1H), 6.15 (m, 2H), 6.24 (m, 2H), 6.99 (dd, 1H, J=11.4 Hz, J=15.0 Hz); ¹³C NMR (100 MHz, CDCl₃): δ 12.9, 13.6, 19.2, 21.7, 2×28.9, 3×30.8, 33.1, 34.2, 3×36.3, 39.6, 3×41.5, 79.9, 120.7, 128.4, 129.6, 129.9, 130.3, 135.5, 137.3, 137.7, 139.1, 151.4, 166.5; MS (ESI) exact mass calculated for C₃₀H₄₃O₂ ([M+H]⁺) 435.3263, found 435.3277.

Animals and dose administration. Rhino mice 6-8 weeks old were orally dosed. The mice were dosed daily. The mice were weighed three times per week, and doses were adjusted weekly based on body weight. The oral formulation was made by mixing each API with Wesson® soybean oil. The oral dose was delivered to the back of the mouth of each mouse. Mice were sacrificed 4 hours after the final oral dose. At sacrifice, the dorsal skin was collected for histological studies.

Comedolytic effect. The extent of the comedolytic effect (i.e., efficacy) was assessed by measuring the average area of the comedones, whereby the smaller the area, the larger the effect and efficacy. The comedone area was determined by histological analysis of tissue sections. Skin was fixed overnight in 4% paraformaldehyde at 4° C. with gentle agitation, and the skin was dehydrated the next day in 100% methanol. Samples were embedded in paraffin and a qty. nine 10μ sections each spaced 150μ apart were prepared from each Rhino mouse. Five of the nine sections were digitally imaged (6× magnification) for comedone analysis using Metamorph Imaging Software (trace function).

The perimeter of each imaged comedone taken from the 5 sections was traced using a Wacom Intuos 3 Graphics Tablet interfaced with the software. The number of pixels representing each individual comedone area was obtained. The mean number of pixels per comedone was obtained for each Rhino mouse. For comedones that were completely healed (i.e., area=0), a pixel value approaching 0 (<10) was assessed. The individual comedone average for each mouse was used to calculate the treatment group mean. Results in the figures herein have been expressed in terms of mean±standard error of the mean.

Preparation of oral retinoid dosing formulations. A concentrated stock solution containing each retinoid was prepared in dichloromethane. The retinoid concentrated stock solution was diluted into ethanol. The concentration of retinoid in that diluted stock solution was determined spectrophotometrically, whereby the molar extinction coefficient=44,340 and λₘₐₓ=350. The purity of the SB-RA, IPE-RA, IB-RA, and t-butyl RA was determined to be greater than 98% by HPLC. A predetermined volume of retinoid-containing solution was added to Wesson® oil, the sample was mixed, and the residual dichloromethane was removed by flushing the oil with argon for 2-3 hours under a vapor hood. The final dose was delivered orally using ≦50 μL of oil.

For treating a human, various therapeutically effective doses and dosing regimens thereof may be determined from the animal data set forth herein using known Allometric Scaling (AS) factors. For example, for a mouse having a body weight of 0.03 kg, the AS factor is around 7 assuming a human body weight of 70 kg.

Predictive dosing ranges have been calculated assuming that for the high end of the oral dose range, the top dose given to the Rhino mouse is corrected for the expected lesser sensitivity of the human and further increased by 0.5 log dose. The low dose is 1×10⁶ lower than the high dose. For the high end of the topical dose, the value was further multiplied by a factor of 20 as humans absorb only about 5% of the dose compared to 100% by the mouse.

Predictive exemplary oral and topical dosing is set forth in Tables 1 and 2, respectively, whereby the dosing ranges are based on known sensitivity to oral all-trans retinoic acid in humans and activity of compounds in the mouse model relative to the activity of all-trans retinoic acid.

TABLE 1

| All-trans | Predictive Exemplary Oral |
|---|---|
| retinoic ester | Dosing of API in Humans |
| SB-RA | 2.4 mcg to 71 mg/kg_{BW}/day |
| IPE-RA | 2.3 mcg to 69 mg/kg_{BW}/day |
| IB-RA | 2.4 mcg to 71 mg/kg_{BW}/day |
| t-butyl-RA | 17 mcg to 498 mg/kg_{BW}/day |
| IA-RA | 0.2 mg to 8.7 g/kg_{BW}/day |

TABLE 2

| All-trans | Predictive Exemplary Topical |
|---|---|
| retinoic ester | Dosing of API in Humans |
| SB-RA | 6.4 ng to 12.8 mg/kg_{BW}/day |
| IPE-RA | 6.2 ng to 12.4 mg/kg_{BW}/day |
| IB-RA | 6.4 ng to 12.8 mg/kg_{BW}/day |
| t-butyl-RA | 45 ng to 90 mg/kg_{BW}/day |
| IA-RA | 78 mcg to 1.6 g/kg_{BW}/day |

1.-7. (canceled)

8. An active pharmaceutical ingredient selected from the group consisting of: 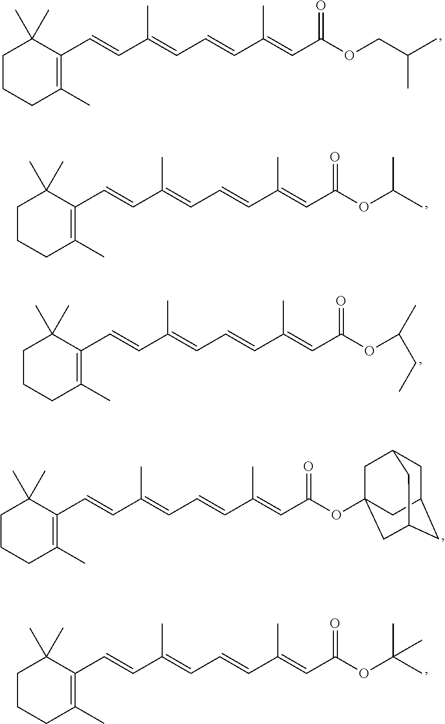
a solvate thereof, and a hydrate thereof.

9. The active pharmaceutical ingredient of claim 8, selected from the group consisting of: 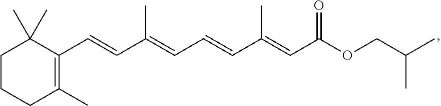
a solvate thereof, and a hydrate thereof.

10. The active pharmaceutical ingredient of claim 8, selected from the group consisting of: 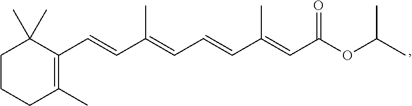
a solvate thereof, and a hydrate thereof.

11. The active pharmaceutical ingredient of claim 8, selected from the group consisting of: 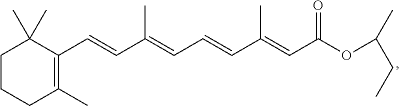
a solvate thereof, and a hydrate thereof.

12. The active pharmaceutical ingredient of claim 8, selected from the group consisting of: 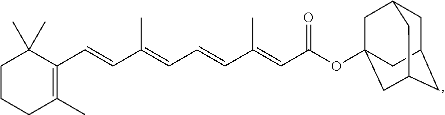
a solvate thereof, and a hydrate thereof.

13. The active pharmaceutical ingredient of claim 8, selected from the group consisting of: 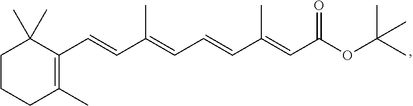
a solvate thereof, and a hydrate thereof.

14. A method of treating acne, psoriasis, ichthyosis, photoaging, skin atrophy caused by corticosteroid treatment or photodamaged skin, or of reducing comedone size, or of increasing epidermal thickness comprising:
orally administering to a human a therapeutically effective dose of an active pharmaceutical ingredient selected from the group consisting of: 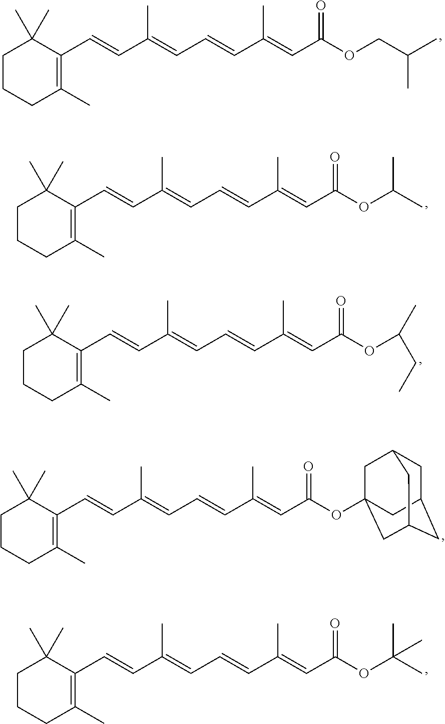
a solvate thereof, and a hydrate thereof.

15. The method of claim 14, wherein the active pharmaceutical ingredient is selected from the group consisting of: 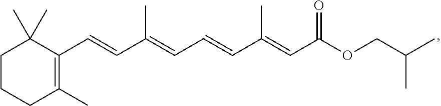
a solvate thereof, and a hydrate thereof.

16. The method of claim 14, wherein the active pharmaceutical ingredient is selected from the group consisting of: 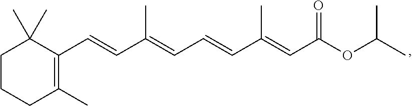
a solvate thereof, and a hydrate thereof.

17. The method of claim 14, wherein the active pharmaceutical ingredient is selected from the group consisting of: 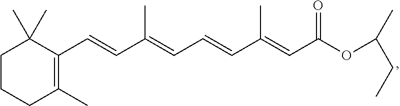
a solvate thereof, and a hydrate thereof.

18. The method of claim 14, wherein the active pharmaceutical ingredient is selected from the group consisting of: 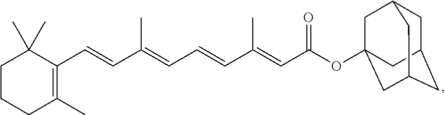
a solvate thereof, and a hydrate thereof.

19. The method of claim 14, wherein the active pharmaceutical ingredient is selected from the group consisting of: 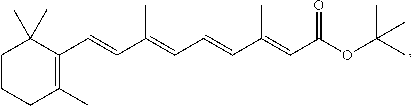
a solvate thereof, and a hydrate thereof.

20. A method of treating acne, psoriasis, ichthyosis, photoaging, skin atrophy caused by corticosteroid treatment or photodamaged skin, or of reducing comedone size, or of increasing epidermal thickness comprising:
topically administering to a human a therapeutically effective dose of an active pharmaceutical ingredient selected from the group consisting of: 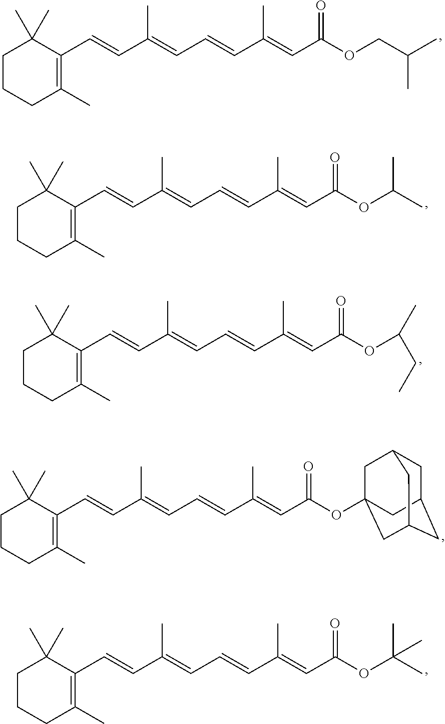
a solvate thereof, and a hydrate thereof.

21. The method of claim 20, wherein the active pharmaceutical ingredient is selected from the group consisting of: 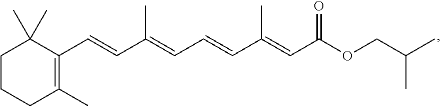
a solvate thereof, and a hydrate thereof.

22. The method of claim 20, wherein the active pharmaceutical ingredient is selected from the group consisting of: 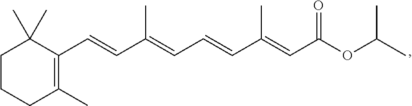
a solvate thereof, and a hydrate thereof.

23. The method of claim 20, wherein the active pharmaceutical ingredient is selected from the group consisting of: 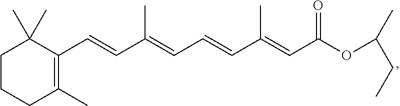
a solvate thereof, and a hydrate thereof.

24. The method of claim 20, wherein the active pharmaceutical ingredient is selected from the group consisting of: 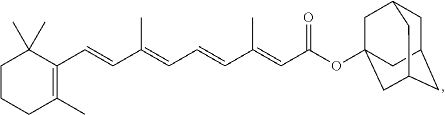
a solvate thereof, and a hydrate thereof.

25. The method of claim 20, wherein the active pharmaceutical ingredient is selected from the group consisting of: 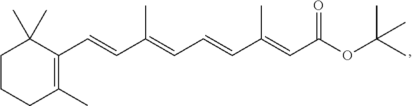
a solvate thereof, and a hydrate thereof.

26. An oral dosage form composition comprising:
a therapeutically effective dose of an active pharmaceutical ingredient selected from the group consisting of: 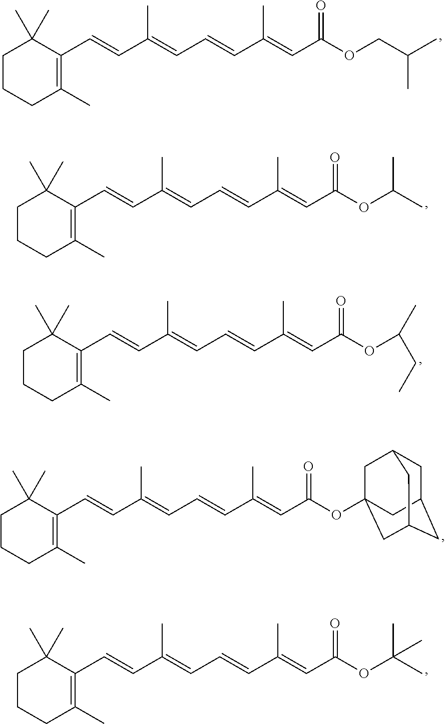
a solvate thereof, and a hydrate thereof; in combination with
a pharmaceutically suitable oral carrier system.

27. The composition of claim 26, wherein the active pharmaceutical ingredient is selected from the group consisting of: 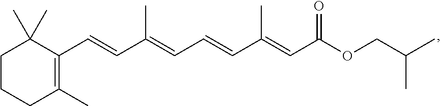
a solvate thereof, and a hydrate thereof.

28. The composition of claim 26, wherein the active pharmaceutical ingredient is selected from the group consisting of: 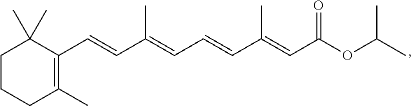
a solvate thereof, and a hydrate thereof.

29. The composition of claim 26, wherein the active pharmaceutical ingredient is selected from the group consisting of: 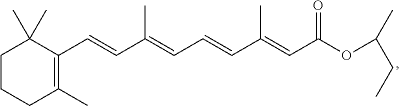
a solvate thereof, and a hydrate thereof.

30. The composition of claim 26, wherein the active pharmaceutical ingredient is selected from the group consisting of: 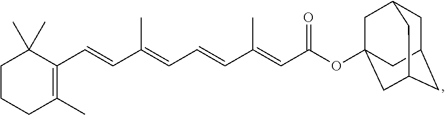
a solvate thereof, and a hydrate thereof.

31. The composition of claim 26, wherein the active pharmaceutical ingredient is selected from the group consisting of: 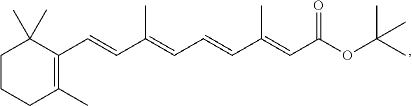
a solvate thereof, and a hydrate thereof.

32. A topical dosage form composition comprising:
a therapeutically effective dose of an active pharmaceutical ingredient selected from the group consisting of: 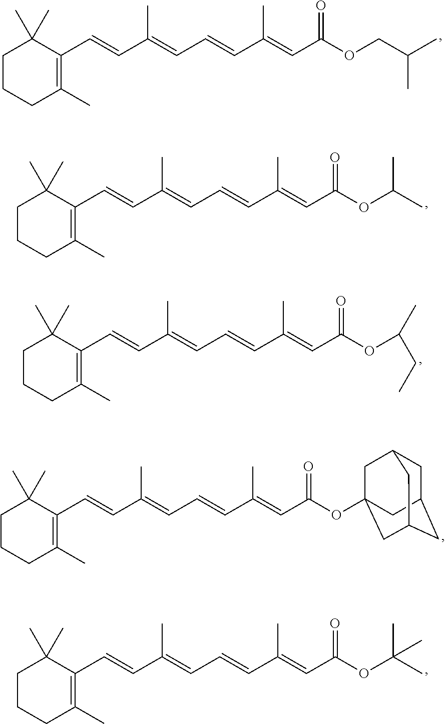
a solvate thereof, and a hydrate thereof;
in combination with a pharmaceutically suitable topical carrier system.

33. The composition of claim 32, wherein the active pharmaceutical ingredient is selected from the group consisting of: 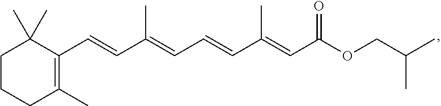
a solvate thereof, and a hydrate thereof.

34. The composition of claim 32, wherein the active pharmaceutical ingredient is selected from the group consisting of: 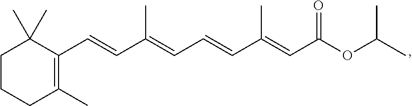
a solvate thereof, and a hydrate thereof.

35. The composition of claim 32, wherein the active pharmaceutical ingredient is selected from the group consisting of: 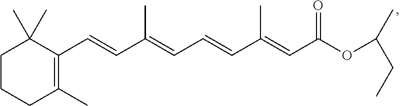
a solvate thereof, and a hydrate thereof.

36. The composition of claim 32, wherein the active pharmaceutical ingredient is selected from the group consisting of: 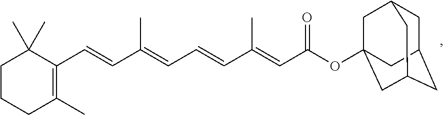
a solvate thereof, and a hydrate thereof.

37. The composition of claim 32, wherein the active pharmaceutical ingredient is selected from the group consisting of: 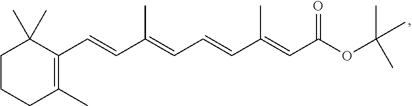
a solvate thereof, and a hydrate thereof.

38. A method of treating acne, psoriasis, ichthyosis, photoaging, skin atrophy caused by corticosteroid treatment or photodamaged skin, or of reducing comedone size, or of increasing epidermal thickness comprising:
orally administering to a human a therapeutically effective dose of an oral dosage form composition comprising a therapeutically effective dose of an active pharmaceutical ingredient selected from the group consisting of: 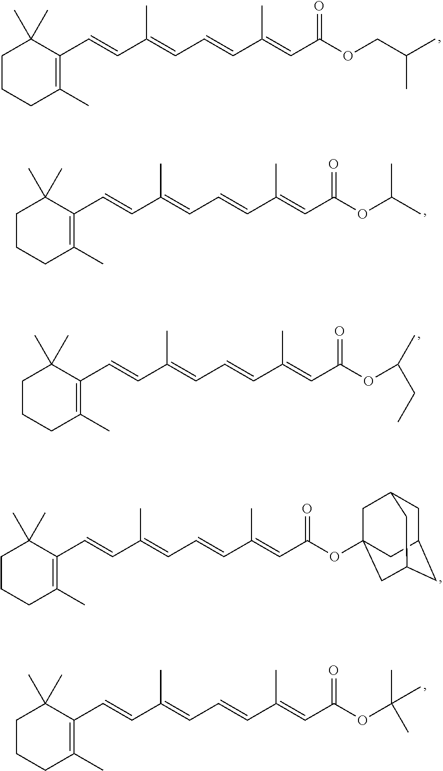
a solvate thereof, and a hydrate thereof; in combination with
a pharmaceutically suitable oral carrier system.

39. The method of claim 38, wherein the active pharmaceutical ingredient is selected from the group consisting of: 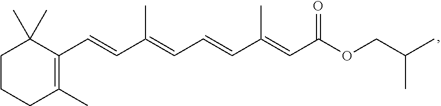
a solvate thereof, and a hydrate thereof.

40. The method of claim 38, wherein the active pharmaceutical ingredient is selected from the group consisting of: 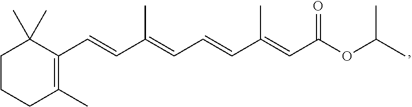
a solvate thereof, and a hydrate thereof.

41. The method of claim 38, wherein the active pharmaceutical ingredient is selected from the group consisting of: 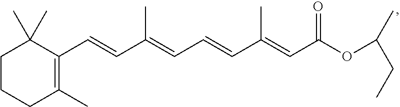
a solvate thereof, and a hydrate thereof.

42. The method of claim 38, wherein the active pharmaceutical ingredient is selected from the group consisting of: 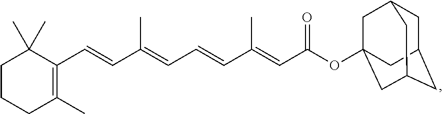
a solvate thereof, and a hydrate thereof.

43. The method of claim 38, wherein the active pharmaceutical ingredient is selected from the group consisting of: 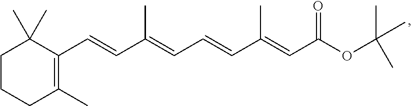
a solvate thereof, and a hydrate thereof.

44. A method of treating acne, psoriasis, ichthyosis, photoaging, skin atrophy caused by corticosteroid treatment or photodamaged skin, or of reducing comedone size, or of increasing epidermal thickness comprising:
topically administering to a human a therapeutically effective dose of a topical dosage form composition comprising a therapeutically effective dose of an active pharmaceutical ingredient selected from the group consisting of: 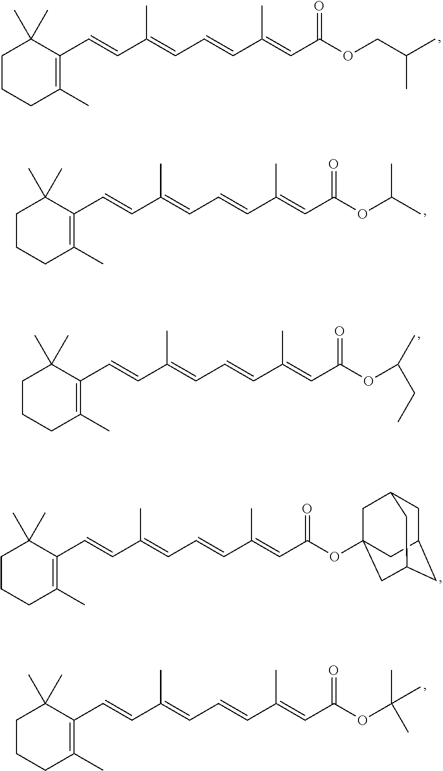
a solvate thereof, and a hydrate thereof; in combination with
a pharmaceutically suitable topical carrier system.

45. The method of claim 44, wherein the active pharmaceutical ingredient is selected from the group consisting of: 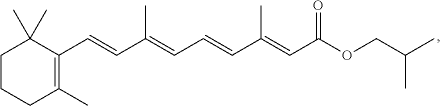
a solvate thereof, and a hydrate thereof.

46. The method of claim 44, wherein the active pharmaceutical ingredient is selected from the group consisting of: 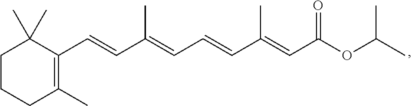
a solvate thereof, and a hydrate thereof.

47. The method of claim 44, wherein the active pharmaceutical ingredient is selected from the group consisting of: 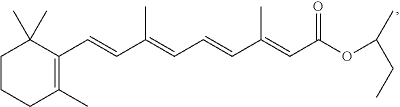
a solvate thereof, and a hydrate thereof.

48. The method of claim 44, wherein the active pharmaceutical ingredient is selected from the group consisting of: 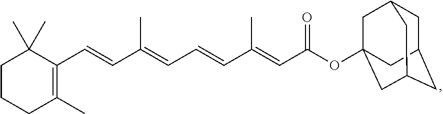
a solvate thereof, and a hydrate thereof.

49. The method of claim 44, wherein the active pharmaceutical ingredient is selected from the group consisting of: 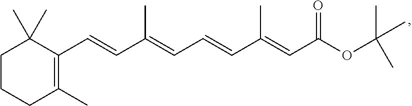
a solvate thereof, and a hydrate thereof.
